(19) United States
(12) Plant Patent Application Publication
(10) Pub. No.: US 2013/0180020 P1
(43) Pub. Date: Jul. 11, 2013

(54) Liriope muscari plant named 'LIRES'

(50) ;

(71) Applicant: Todd Layt, Clarendon (AU)

(72) Inventor: Todd Layt, Clarendon (AU)

(21) Appl. No.: 13/374,696

(22) Filed: Jan. 9, 2012

(51) Int. Cl. A01H 5/00

(52) U.S. Cl. PLT/263.1

(57) ABSTRACT

‘LIRES’is a distinctive variety of Liriope muscari which is characterized by the combination of medium plant height, dense foliage, pale yellow immature foliage with numerous inflorescences with violet flowers.

### LATIN NAME OF THE GENUS AND SPECIES

The Latin name of the genus and species of the novel variety disclosed herein is Liriope muscari.

### VARIETY DENOMINATION

The inventive variety of Liriope disclosed herein has been given the variety denomination ‘LIRES’.

### BACKGROUND OF THE INVENTION

The present invention relates to a new and distinct perennial variety of Liriope muscari, which has been given the variety denomination of ‘LIRES’. Its market class is that of an ornamental flowering perennial. ‘LIRES’ is intended for use in landscaping and also in garden containers.

Parentage: The Liriope muscari variety ‘LIRES’ was finally selected in 2007 in an Australian nursery in the state of New South Wales following a selection process carried out from 2005 to 2007 involving approximately 20000 seedlings of openly pollinated Liriope muscari (unpatented) production stock which displayed a varying degree of defective chloroplast, commonly referred to as leaf variegation. ‘LIRES’ was selected for the combination of dense foliage and early spring flushes of white foliage that persists for several months in spring before turning green and a vigorous flowering habit.

Asexual Reproduction: ‘LIRES’ was first propagated asexually by division in 2008 in the state of New South Wales, Australia and has since been asexually propagated through several subsequent generations. The distinctive characteristics of the inventive ‘LIRES’ variety are stable from generation to generation; clones of the variety produced by asexual reproduction maintain the distinguishing characteristics of the original plant.

The immature foliage of ‘LIRES’ is almost completely void of functioning chloroplast which is expressed as very pale yellow, almost white, new growth that emerges in early spring whereas the parent, an unpatented and uncommercialized strain of Liriope muscari with defective chloroplast, emerges from dormancy with a far less degree of defective chloroplast in the immature foliage, rendering it undesirable for commercial production.

### SUMMARY OF THE INVENTION

‘LIRES’ is a distinctive variety of Liriope muscari which is characterized by the combination of medium plant height, dense foliage, pale yellow immature foliage with numerous inflorescences containing numerous violet flowers.

### BRIEF DESCRIPTION OF THE FIGURES

FIG. 1 shows two mature ‘LIRES’ plants, approximately 12 months of age, grown in a 140 mm nursery pot.

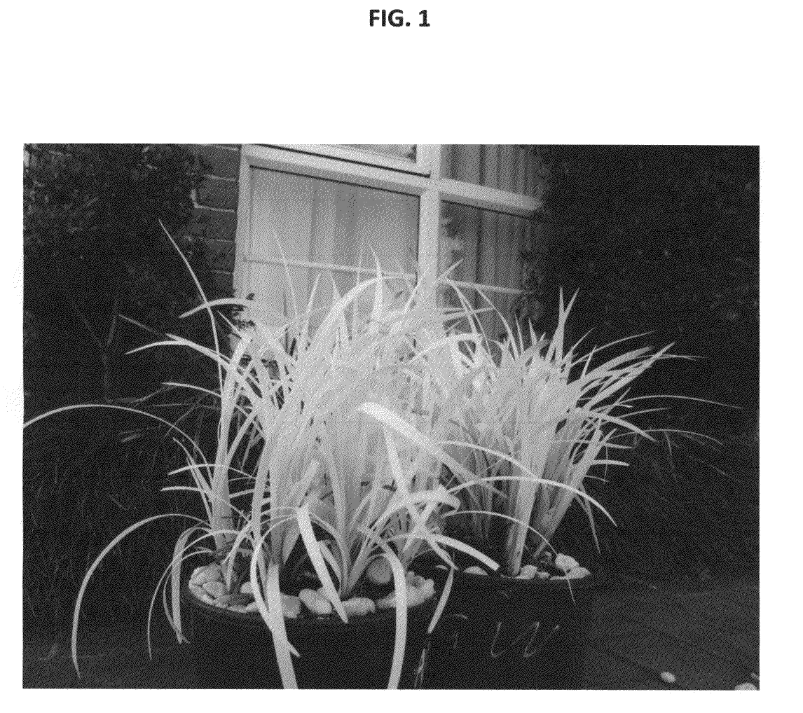

FIG. 2 shows a close up view of the immature foliage

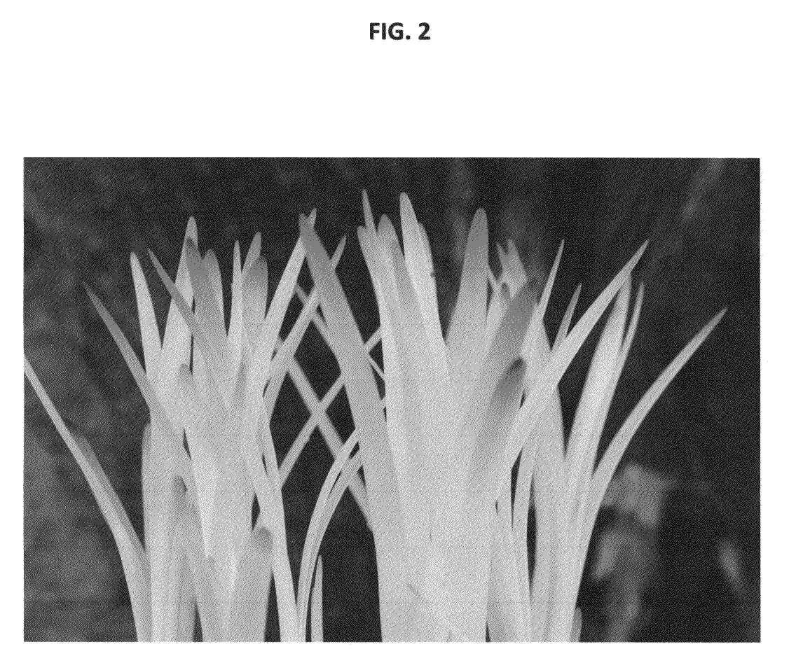

### BOTANICAL DESCRIPTION OF THE PLANT

The following is a detailed botanical description of a new and distinct variety of a Liriope muscari ornamental plant known as ‘LIRES’. Plant observations were made on plants grown in New South Wales, Australia. Unless indicated otherwise, the descriptions disclosed herein are based upon observations made in February 2011 of mature ‘LIRES’ plants, approximately 12 months of age, grown in 140 mm nursery pots.

Those skilled in the art will appreciate that certain characteristics will vary with older or, conversely, younger plants. ‘LIRES’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1986 edition. Note that generic color descriptions such as ‘pink’ do not exist in the R.H.S. charts and the corresponding R.H.S. colors are quoted.

‘LIRES’ is a selection of Liriope muscari, an herbaceous perennial, that is characterized by pale yellow immature foliage that emerges in spring and vigorous flowering habit in late summer and early autumn.

These features and other characteristics are apparent from the description provided below.
Growth habit, dimensions and color:
Plant description:
   Plant habit.—Rhizomatous herbaceous perennial with slightly arching leaves.
   Height.—18 cm at maturity.
   Width.—20 cm at maturity.
   Bloom period.—Summer.
   Hardiness.—USDA Zone 6 to 10.
   Environmental tolerances.—Prefers to be grown in partial shade. Drought tolerant once established; moderate to good recovery with watering after severe wilting. Tolerates a wide range of soil types from sandy loam to loamy clay.
   Pest and disease susceptibility or resistance.—In common with the species, none of note.
   Propagation.—Propagation is accomplished through division of rhizomes.
   Time to initiate roots.—Approximately 2.5 weeks.
   Crop time.—Depending on latitude of and microclimate of growing location, a rooted cutting requires approximately 3 months with an additional five to seven months needed to produce a mature and marketable 14-centimeter container.
Stem:
   Branching and habit.—Leaves arising directly from crown of the plant; acaulescent.
Foliage:
   Type.—Evergreen.
   Shape.—Linear.
   Division.—Simple.
   Apex.—Acute.
   Base.—Truncate.
   Venation.—Parallel.
   Vein color.—Adaxial and abaxial surfaces indistinguishable from the leaf surface.
   Margins.—Entire.
   Arrangement.—Alternate.
   Attachment.—Sessile.
   Texture.—Glossy and coriaceous.
   Surfaces (adaxial surface).—Glabrous at all stages.
   Surfaces (abaxial surface).—Glabrous at all stages.
   Mature leaf dimensions.—average length 21.4 cm, average width 9.5 mm.
   Leaf color (adaxial surface).—Juvenile: yellow 4D with apex close to green 137B; mature: green 137B.
   Leaf color (abaxial surface).—Juvenile: yellow 4D with apex close to green 137B; mature: green 137B.
   Petiole.—Absent; sessile.
Roots: The roots of ‘LIRES’ are freely-branched, colored white, fleshy and thick with terminate tubers, similar to other Liriope muscari.
Inflorescence: The inflorescence is a raceme consisting of approximately 80 to 120 pedicellate, simple flowers. Peduncle length ranges from 14 to 20 cm and the raceme position is at or slightly above the foliage at anthesis. Peduncle color is yellow green RHS 146A at the base, transitioning to approximately purple 79A at mid-peduncle and further transitioning to violet 87B at the raceme. Pedicels vary in length from minute to up to 5 mm long. Pedicel color is violet RHS 85D. Raceme length ranges from 8 to 10 cm. Approximately 12 to 15 inflorescence present at peak flower.
Buds: Obovate flower buds are approximately 2.5 to 3 mm in length and 1.5 mm in width. Color is violet approximately RHS 85A.
Flowers: The flowering season is late summer to autumn. Perianth of 6 unfused segments has a diameter of approximately 5 to 6 mm when fully opened. Mature petal color is violet RHS 85C. Margins entire. Apex is obtuse or rounded. Anthers are yellow RHS 6D and stigma is violet RHS 85B. Flowers are persistent and non-fragrant.
Fruit and seeds: Globose berries, approximately 7 mm in diameter, each containing one seed. Berries are glaborous, shiny and have a color that approximates to green 139A when immature and maturing to black 202A late in the season.
Comparison of LIRES with other varieties of Liriope muscari: ‘LIRES’ is similar to the seed and pollen parent in many characteristics. However, ‘LIRES’ has a short-medium plant height (approximately 18 cm) whereas Liriope muscari (unpatented) has a tall plant height that is highly variable, ranging from approximately 30 cm up to 40 cm. ‘LIRES’ should be compared to Liriope muscari ‘LIRBLONDE’ (AU PBR 2008/310, unpatented in the US) in that both are of comparable size and habit and both have pale yellow immature foliage. However, ‘LIRES’ is a larger plant with a better flower habit. ‘LIRES’ has a mature plant height of 18 cm whereas ‘LIRBLONDE’ of similar age have an average plant height of 11 cm. An important distinguishing characteristic of ‘LIRES’ is that the claimed plant has a significantly more vigorous flower habit. When comparing plants of similar age, approximately 12 months old, ‘LIRES’ displays 12 to 15 inflorescence whereas ‘LIRBLONDE’ has no inflorescence present. The combination of dense foliage, medium height, pale yellow immature foliage and vigorous flowering habit makes ‘LIRES’ a desirable ornamental plant suited for mass production for pot and landscape use.

That which is claimed is:

1. A new and distinct variety of Liriope muscari plant named ‘LIRES’, substantially as described and illustrated herein.
